(19) United States
(12) Patent Application Publication
(10) Pub. No.: US 2018/0161252 A1
(43) Pub. Date: Jun. 14, 2018

(54) MICRONEEDLE DELIVERY SYSTEM AND METHOD

(71) Applicant: University Medical Pharmaceuticals Corp., Irvine, CA (US)

(72) Inventors: Raymond Joseph Francis, Irvine, CA (US); David Bardin, Irvine, CA (US); Gregory Lee Hunt, Irvine, CA (US)

(21) Appl. No.: 15/821,314

(22) Filed: Nov. 22, 2017

Related U.S. Application Data
(60) Provisional application No. 62/425,987, filed on Nov. 23, 2016.

(51) Int. Cl. A61K 8/02; A61K 8/73; A61K 8/34; A61Q 19/08; A61M 37/00

(52) U.S. Cl. CPC A61K 8/0233; A61K 8/735; A61K 8/732; A61K 8/345; A61M 2037/0023; A61M 37/0015; A61K 2800/87; A61M 2037/0061; A61Q 19/08

(57) ABSTRACT

A microneedle device suitable for application to the skin and methods for using the same. The microneedles are made of one or more biocompatible polymers that optionally provide a cosmetic benefit when dissolved within the skin. Optionally, the microneedles also may be used to deliver other active ingredients to the skin. The device is characterized by a microneedle layer, an intermediate layer of a liquid-soluble polymer, and an outer, liquid-permeable layer that is in fluid communication with the intermediate layer. In use, the device is applied to the skin such that the microneedles penetrate the skin surface. A liquid (e.g., water) is applied to the outer layer, causing dissolution of the intermediate layer, thereby detaching the microneedles within the skin from the device body. The remainder of the device is then removed and discarded.

### CROSS-REFERENCE TO RELATED APPLICATIONS

This application claims priority to U.S. provisional patent application, Ser. No. 62/425,987, filed Nov. 23, 2016. Priority to the provisional patent application is expressly claimed, and the disclosure of the provisional application is hereby incorporated herein by reference in its entirety and for all purposes.

### FIELD

The disclosed embodiments relate generally to improved microneedle devices for application to the skin, which optionally incorporate therapeutics, and methods for producing the same.

### BACKGROUND

Microneedle arrays are used as transdermal drug-delivery systems and to deliver polymers directly to the skin for cosmetic applications. Biodegradable microneedles are commonly used. Existing devices provide the biodegradable microneedles attached to a patch having a substrate layer that contacts the skin. In use, the substrate layer or patch is applied to the skin and pressure is applied which causes the microneedles to pierce the stratum corneum. One disadvantage of these devices is that the patch must remain affixed to the skin while the microneedles dissolve within the underlying skin layers. Microneedle dissolution may take several hours to a day or more, depending upon the specific microneedle composition. It is often inconvenient, unsightly, and/or uncomfortable for the user to wear the device for this extended period of time. Thus, there is a need to provide a biocompatible/biodegradable microneedle device which can effectively deliver the microneedles across the stratum corneum and be removed within a short period of time without affecting the performance of the device/microneedles.

### SUMMARY

The present disclosure provides a microneedle device suitable for application to the skin. Various embodiments and features of the microneedle device are described below.

In one aspect, the present disclosure provides a device having: (a) a first layer comprising a plurality of biocompatible microneedles, the microneedles having a tip region and a base region, (b) a second layer comprising a water soluble polymer affixed to the base region of the first layer, and (c) a third layer comprising a water-permeable portion in fluid communication with the second layer.

In some embodiments, the first layer further comprises a substrate layer that is contiguous with the microneedles and attached at the base region. In other embodiments, the first layer lacks a substrate layer and the microneedles are attached at the base region to the second layer.

In some embodiments, the microneedles contain at least one polymer selected from the group consisting of pullulan, hyaluronic acid (HA), polylactic acid (PLA), polyglycolic acid (PGA), poly(lactic-co-glycolic acid) (PLGA), cellulose, sodium carboxymethyl cellulose (SCMC), hydroxyethyl cellulose (HEC), hydroxypropyl cellulose (HPC), hydroxypropyl methylcellulose (HPMC), amylopectin (AMP), silicone, polyvinylpyrolidone (PVP), polyvinyl alcohol (PVA), poly(vinylpyrrolidone-co-methacrylic acid) (PVA-MAA), polyhydroxyethylmethacrylate (pHMEA), polyethlene glycol (PEG), polyethylene oxide (PEO), polyacrylic acid, chrondroitin sulfate, dextrin, dextran, maltodextrin, chitin, chitosan, mono- and polysaccharides, galactose, and maltose. In particular embodiments, the microneedles comprise hyaluronic acid or a mixture of hyaluronic acid and pullulan.

In some embodiments, the microneedles also contain at least one sugar alcohol (e.g., mannitol, sorbitol, and xylitol).

In some embodiments, the microneedles also contain an active ingredient.

In some embodiments, the second layer contains a polymer selected from the group consisting of pullulan, PVP, PGA, PLGA, PLA, and mixtures thereof.

Optionally, the third layer has an overhang region that extends beyond an outer dimension of the first layer and the second layer, and wherein the overhang region further comprises an adhesive on a skin-facing surface.

In some embodiments, the microneedles contain 1.0%-7.5% hyaluronic acid (HA), 2.5%-15% pullulan, and 0.5%-5.0% mannitol. The HA may be crosslinked or uncrosslinked. Optionally, uncrosslinked HA may be present at about 3%-6%. Optionally, crosslinked HA may be present at about 1%-4%. Optionally, pullulan is present in a concentration of about 3%-12%, including 3%-6%, 5%-10%, and 4%-12%.

In other embodiments, the microneedles contain a mixture of low molecular weight HA (“low MW HA”) and high molecular weight HA (“high MW HA”). In some embodiments, the low MW HA is present in a concentration of about 0.25-5%, including for example, 1.0-3.0% (e.g., about 0.25%, 0.5%, 0.75%, 1.0%, 1.25%, 1.5%, 1.75%, 2.0%, 2.25%, 2.5%, 2.75%, 3.0%, 3.25%, 3.5%, 3.75%, 4.0%, 4.25%, 4.5%, 4.75%, and 5%) and the high MW HA is present in a concentration of about 0.25%-3.0%, including for example, about 0.25%, 0.5%, 0.75%, 1.0%, 1.25%, 1.5%, 1.75%, 2.0%, 2.25%, 2.5%, 2.75%, and 3.0%.

In some embodiments, the microneedles have a diamond shape.

In another aspect, the present disclosure provides a method for delivering a cosmetic polymer to the skin for any suitable purpose, including purposes described herein, by: (a) providing a three-layer microneedle-containing device as described herein, (b) applying the device to a skin region of a subject, (c) applying skin-directed pressure to the third layer sufficient to cause the plurality of microneedles to puncture a stratum corneum of the skin region, (d) applying a liquid (e.g., water, an aqueous solution, or a solution containing an organic solvent) to the third layer to cause the second layer to dissolve, (e) waiting for a period of time until the second layer is substantially completely dissolved (e.g., less than 5, 10, 15, 20, 25, 30, 45, 60, or 120 minutes), and (f) removing the device from the skin region such that the microneedles remain embedded in the skin region.

In another aspect, the present disclosure provides a device having a plurality of biocompatible microneedles comprising 1.0%-7.5% hyaluronic acid (HA) and 2.5%-15% pullulan. Optionally, the microneedles also contain a sugar alcohol (e.g., mannitol) which may be present in any suitable concentration including, for example, about 0.5%-5.0%. The HA may be crosslinked or uncrosslinked, and may comprise only low MW HA, only high MW HA, or a mixture thereof, as described herein.

In another aspect, the present disclosure provides a device having a plurality of biocompatible microneedles comprising 1.0%-7.5% hyaluronic acid (HA) and 0.5%-5.0% sugar alcohol (e.g., mannitol). The HA may be crosslinked or uncrosslinked, and may comprise only low MW HA, only high MW HA, or a mixture thereof, as described herein.

By “microneedles” is meant a plurality of protrusions, as described herein, and have a height (h), measured from the inner surface of the intermediate layer, or the inner surface of the substrate layer, if present, to the tip of the microneedle, of about 100 μm-1,500 μm, including for example about 300 μm-1,000 μm, or about 400 μm-800 μm, including about 100 μm, 200 μm, 300 μm, 400 μm, 500 μm, 600 μm, 700 μm, 800 μm, 900 μm, 1,000 μm, 1,100 μm, 1,200 μm, 1,300 μm, 1,400 μm, and 1,500 μm. In other embodiments, the aspect ratio (i.e., ratio of height to base) of the microneedles is about 1.0-4.0, including about 1.5-3.5, and 2.0-3.0, including, for example, about 1.0, 1.25, 1.5, 1.75, 2.0, 2.25, 2.5, 2.75, 3.0, 3.25, 3.5, 3.75, and 4.0. In some embodiments, the microneedles have absolute dimension for the base of about 50 μm, 100 μm, 150 μm, 200 μm, 250 μm, 300 μm, 350 μm, 400 μm, 450 μm, 500 μm, 550 μm, or 600 μm. In other embodiments, the microneedles have an absolute dimension (height to base) of about 400:200 μm, 600:300 μm, or 800:400 μm. Microneedles may be formed into any suitable shape including, for example, conical, diamond, tetrahedral, and pyramidal shapes.

By “pullulan” is meant a polysaccharide polymer consisting of maltotriose units in which the three glucose units in maltotriose are joined by an α-1,4 glycosidic bond and consecutive maltotriose units are joined to each other by an α-1,6 glycosidic bond. In some embodiments, pullulan has an average molecular weight of about 5,000-20,000 Da, including about 7,500-15,000 Da (e.g., about 5,000, 6,000, 7,000, 8,000, 9,000, 10,000, 11,000, 12,000, 13,000, 14,000, 15,000, 16,000, 17,000, 18,000, 19,000, and 20,000 Da, or more).

“Proximal” or “inner,” when referring to a microneedle device, refers to the layer or surface of the device closest to the subject/user (e.g., the skin-facing surface). When applied to the skin, the proximal/inner surface of the device is the surface that first contacts the skin and initially contains the microneedle array.

“Distal” or “outer,” when referring to a microneedle device, refers to the layer or surface of the device that is farthest from skin relative to the proximal/inner surface. When applied to the skin, the distal surface may form a protective, occlusive, semi-occlusive, permeable, semi-permeable, or non-permeable covering over all or just a portion of the proximal/inner layer. It is understood that the distal/outer layer may be larger in area than the proximal/inner layer and therefore may contact the skin towards its edges. Optionally, the distal/outer layer may have an inwardly-facing adhesive adapted to hold the inner layer against the skin. The inwardly-facing adhesive may circumnavigate the entire perimeter of outer layer or just a portion of the perimeter. Further, the adhesive may be a continuous adhesive strip or discontinuous dots or patches of adhesive about the perimeter.

When referring to relative polymer concentrations (i.e., percentages), for convenience, reference is made to the polymer concentration in solution prior to molding and drying.

### BRIEF DESCRIPTION OF THE DRAWINGS

FIG. 1 is schematic cross-section of an exemplary microneedle device.

FIGS. 2A-2B are schematic plan views of microneedle array configurations.

FIGS. 2C-2D are schematic plan views of the dissolution profiles of microneedle array configurations.

FIG. 3 is a schematic of a diamond microneedle design.

### DETAILED DESCRIPTION OF THE PREFERRED EMBODIMENTS

The preferred embodiments generally provide microneedle devices for application to the skin. The devices generally provide an array of dissolvable microneedles which deliver polymeric compositions beneath the skin surface in order to reduce or eliminate fine lines, wrinkles, stretch marks, scars, cellulite, and other skin imperfections, or to smooth, texture, tighten, and/or hydrate the skin. Optionally, the microneedles may contain and deliver to the skin one or more therapeutic agents or compounds other than the polymeric formulation of the dissolvable microneedles. The microneedle device may be applied to the skin on any part of the body for which treatment is desired including, for example, the face (cheeks, forehead, and/or periorbital region) neck, dëcolletage, back of hands, armpits, arms, and legs. The device may have any shape and, generally, the shape varies based on the desired application site on the human body.

Microneedle Device

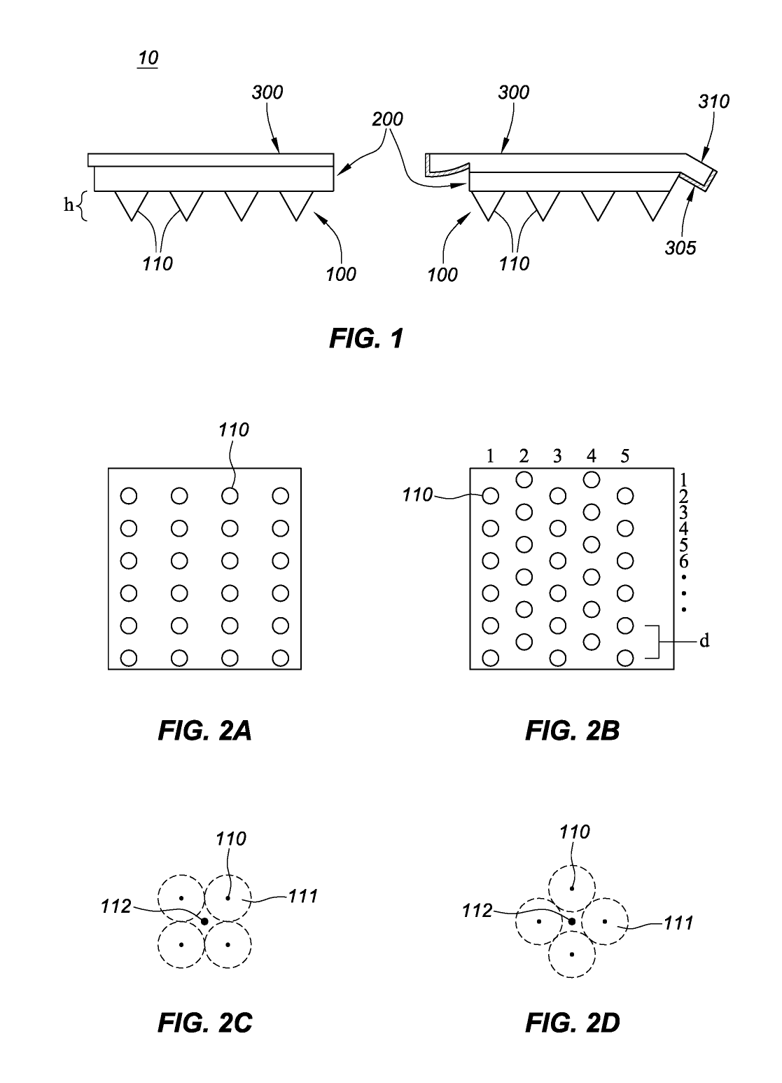

In one aspect, the present disclosure provides a three-layer microneedle device 10, as illustrated in FIG. 1. Generally, the device consists of an inner/proximal layer 100 that contains a plurality of microneedles 110 (e.g., a microneedle array), a dissolvable intermediate substrate layer 200, and a permeable outer/distal backing layer 300. Each layer is described in more detail below.

Inner Layer Comprising Microneedles

The inner layer comprises dissolvable microneedles that are designed to be pressed into the skin of the user. The microneedles 110 may be present in an array (i.e., a regular and ordered pattern) or randomly distributed on the inner surface of this first layer. The microneedles may be arranged in a parallel array (FIG. 2A) or an offset array (FIG. 2B). Arrays are generally constructed such that the microneedles 110 fall within regularly spaced rows and columns, forming a grid-like pattern. For parallel arrays, microneedles 110 in adjacent row and columns are disposed next to each other such that, for any given column, a microneedle 110 is present in every row. For offset arrays, microneedles 110 in adjacent columns are out-of-phase such that for any given column, a microneedle 110 is present in only every second in adjacent columns. For example, as illustrated in FIG. 2B, odd-numbered rows have a microneedle only in the even-numbered columns and even-numbered rows have a microneedle 110 only in the odd-numbered columns.

In one embodiment, the microneedles 110 are present in an offset array in which the microneedles in each column are offset from adjacent columns by about 50% of the distance “d” between microneedles 110, as illustrated in FIG. 2B. The offset array provides significant advantages over the parallel array for swellable microneedles 110 made from polymers designed as skin fillers (i.e., to smooth the exterior surface of the skin and/or remove lines and/or wrinkles). Microneedles 110, regardless of shape (e.g., conical and pyramidal) generally dissolve to form a polymer halo 111 within the skin that is substantially circular with higher polymer concentrations closer to halo center/microneedle body. As illustrated in FIG. 2C, dissolution of microneedles 110 in a parallel array form a void area 112 characterized by significantly less or no dissolved polymer, depending upon the microneedle 110 size and spacing. The presence of void areas 112 can cause the skin to take on a lumpy or pitted appearance. In contrast, dissolution of microneedles 110 in offset arrays, as illustrated in FIG. 2D, produce a significantly smaller void area 112, thereby producing a smoother and more desirable effect. Additionally, offset arrays produce a more even dispersion of any cosmetic or therapeutic agents that may be delivered using the microneedle platform.

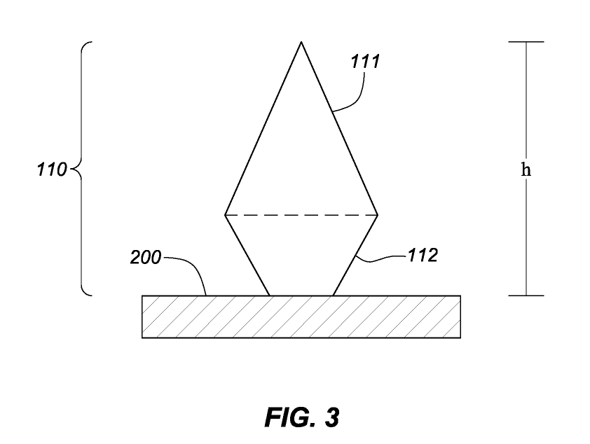

The individual microneedles 110 may have any appropriate shape and dimension. For example, the microneedles 110 may be shaped as pyramids, prongs, diamonds, and cones. A review of various microneedle shapes and designs is provided in Prausnitz et al, Adv. Drug Deliv. Rev. 56:581-587, 2004 and WO 2011/076537, each of which is hereby incorporated by reference in its entirety. In one embodiment, the microneedles 110 have a diamond shape, as illustrated in FIG. 3. A diamond shaped microneedle is characterized by an upper body portion 111 and a lower body portion 112 that may or may not be symmetrical. The base may be the intermediate substrate layer 200, as illustrated in FIG. 3, or a contiguous substrate layer joining the microneedles in the base region which is itself affixed to the intermediate substrate layer 200. Although FIG. 3 illustrates the diamond-shaped microneedle 110 as having a conical body 111, it is understood that the body 111 may be any suitable shape including, for example, pyramidal. The lower body portion 112 may have any convenient dimension including for example, less than 5%, 10%, 15%, 20%, 25%, 30%, 35%, 40%, 45%, or 50% of the total height (h) of the microneedle 110. A microneedle 110 having a diamond shape may more rapidly detach from the device, and specifically upon the dissolution of intermediate substrate layer 200 because the lower body portion provides a smaller point of attachment between the base region of the microneedle 110 and the intermediate substrate layer 200 compared to other microneedle 110 configurations.

The microneedles 110 may have any height suitable to application to the skin. Microneedle 110 height may be selected to reach or target specific depths or skin layers including for example, the epidermis, dermis, and subcutaneous tissue, or specific boundary regions such as the dermal/epidermal junction.

The inner layer 100 may be continuous or discontinuous. Continuous inner layers have a substrate layer that is usually relatively thin and reversibly or irreversibly bonded to the intermediate layer 200 on its distal face and contains a plurality of microneedles 110 extending from its proximal face. A discontinuous inner layer 100 contains only the plurality of microneedles 110 which are directly supported by, and attached to the intermediate layer 200. It is recognized that the substrate layer of a continuous inner layer also may serve as an intermediate substrate layer 200, when no separate intermediate substrate layer 200 is included, provided that the polymer composition of that substrate layer meets the other requirements of an intermediate substrate layer 200, as described herein.

Inner layer 100 may contain 10-10,000 microneedles/cm² or more including, for example, at least about 50, 100, 250, 500, 750, 1000, 2,500, 5,000, 7,500, or 10,000 microneedles/cm².

Microneedles 110 may be formed from any suitable biocompatible and biodegradable polymer (e.g., that dissolves in the skin). Suitable soluble polymers include, for example, pullulan, hyaluronic acid (HA), polylactic acid (PLA), polyglycolic acid (PGA), poly(lactic-co-glycolic acid) (PLGA), cellulose, sodium carboxymethyl cellulose (SCMC), hydroxyethyl cellulose (HEC), hydroxypropyl cellulose (HPC), hydroxypropyl methylcellulose (HPMC), amylopectin (AMP), silicone, polyvinylpyrolidone (PVP), polyvinyl alcohol (PVA), poly(vinylpyrrolidone-co-methacrylic acid) (PVA-MAA), polyhydroxyethylmethacrylate (pHMEA), polyethlene glycol (PEG), polyethylene oxide (PEO), polyacrylic acid, chrondroitin sulfate, dextrin, dextran, maltodextrin, chitin, chitosan, mono- and polysaccharides, galactose, and maltose, and mixtures thereof.

In some embodiments, the microneedles are formed from, or may include (i.e., in combination with one or more other polymers) hyaluronic acid (“HA”). In some embodiments, the HA is cross-linked including, for example, disulfide crosslinked HA. HA may be disulfide crosslinked by any suitable method and derivatization scheme. Exemplary disulfide crosslinked HA includes, for example, dihydrazide-functionalized HA (see, for example, U.S. Pat. No. 5,616,568 and Shu et al., Biomacromolecules, 3:1304-1311, 2002; each of which is hereby incorporated by reference in its entirety). The specific form of HA, and its concentration, are selected based on the desired properties of the microneedle. In some embodiments, the HA portion may comprise HA of a single molecular weight or multiple molecular weights, and/or may contain one or more forms of derivatized and/or underivatized HA. Suitable HAs include, for example, “low molecular weight” HAs having an average MW of about 200 kDa-<1 MDa (e.g., about 250 kDa, 300 kDa, 350 kDa, 400 kDa, 450 kDa, 500 kDa, 600 kDa, 700 kDa, 750 kDa, 800 kDa, 900 kDa, and 950 kDa) and “high molecular weight” HAs having an average MW of about 1-3 MDa (e.g., about 1.0 MDa, 1.1 MDa, 1.2 MDa, 1.3 MDa, 1.4 MDa, 1.5 MDa, 1.6 MDa, 1.7 MDa, 1.8 MDa, 1.9 MDa, 2.0 MDa, 2.2 MDa, 2.4 MDa, 2.6 MDa, 2.8 MDa, and 3.0 MDa).

In some embodiments, the microneedles comprise pullulan and HA in any concentration or combination of concentrations described herein.

In one embodiment, the inner/microneedle layer is discontinuous and affixed to the intermediate substrate layer. The microneedles are first formed, as described herein, and the intermediate substrate layer is affixed thereto. In another embodiment, the inner/microneedle layer is continuous in which the microneedles are contiguously formed with a thin substrate layer of the same or a different polymer. The substrate layer may be water-soluble, water-insoluble, or partially water-soluble.

Intermediate Substrate Layer

The intermediate substrate layer 200 is adapted to support the microneedles 110 and provide sufficient structural rigidity, in combination with backing layer 300, to effectively transfer and apply force to the microneedles 110 so that the microneedles may pierce the stratum corneum. In one embodiment, the intermediate substrate layer 200 is formed from a water-soluble polymer or mixture of water-soluble polymers. Preferably, substantially all of the intermediate substrate layer 200 is formed from a water-soluble polymer or mixtures thereof. The intermediate substrate layer 200 is configured such that, upon the application of water and dissolution of the layer, the inner layer 100 is completely disengaged from any remainder of the device. Any aqueous residue resulting from intermediate substrate layer dissolution may be carefully wiped away by the user so as not to disturb the microneedles embedded within the skin.

In some variations, the inner layer 100 is discontinuous such that the microneedles 110 are supported and attached to only the intermediate substrate layer 200. The intermediate layer may have any suitable thickness, depending upon the structural properties of the polymer used and those of the backing layer 300 to which the intermediate substrate layer 200 is attached. Conveniently, the thickness of the intermediate substrate layer 200 is about 5-30 mils (e.g., about 10-20 mils) including, for example, about 5 mils, 10 mils, 15 mils, 20 mils, 25 mils, or 30 mils.

Suitable water-soluble polymers include, for example pullulan, PVP, PGA, PLGA, PLA, and mixtures thereof. In one embodiment, intermediate substrate layer 200 is at least 50%, 60%, 70%, 75%, 80%, 85%, 90%, 95%, or 100% pullulan. In addition to water solubility, the selection of intermediate substrate layer 200 polymers, polymer molecular weight and degree of cross-linking, and thickness should be made to maintain the flexibility of the intermediate substrate layer 200 in order that the layer, and the device as a whole, can be easily handled and contoured to the application site on the skin surface without significant brittleness, cracking, or breaking. Optionally, the intermediate substrate layer 200 may contain small amounts of modifiers and other reagents including, for example, surfactants (e.g., TEG1000 and Tween) and/or plasticizers (e.g., glycerine).

In some embodiments, the intermediate substrate layer 200 comprises, or is made exclusively from pullulan. Alternatively, the intermediate substrate layer 200 comprises, or is made exclusively from PVP-PVA copolymers (e.g., Luvitec VA-64), chitosan, HA, and mixtures thereof alone or in combination with pullulan. When HA is included in the intermediate substrate layer 200, low molecular weight and uncrosslinked HA is particularly useful.

Backing Layer

Backing layer 300 is non-occlusive, water-permeable, and adapted to support intermediate layer 200. Backing layer 300 may be formed from any suitable web, mesh, or woven material including, for example, pressed, woven and non-woven cellulose fibers, PLA webs, and membrane filters (e.g., porous films of polyester, nylon, and the like).

Backing layer 300 may be substantially the same dimension as intermediate layer 200, or it may overhang intermediate layer 200 in one or dimension. In one embodiment illustrated in FIG. 1, backing layer 300 has an overhang region 310 that extends beyond the dimension of intermediate layer 200. Optionally, overhang region 310 has an adhesive 305 (e.g., a pressure-sensitive adhesive) on the skin-facing surface. The overhang region 310 may or may not be water-permeable and may be made from the same or different material than the remainder of the backing layer 300 that overlays the intermediate layer 200.

Microneedle Device Use and Application

The microneedle devices described herein provide a rapid and effective system for applying the microneedles to the skin. The microneedles may be released after implantation without the user waiting for the microneedles to dissolve. In use, the microneedle device is placed against the skin in the target location for which treatment is desired. The microneedles are caused to penetrate the stratum corneum by the application of pressure to the backing layer sufficient to drive the microneedles into the skin to substantially their full depth. Optionally and when provided, the device may be held in place by an adhesive portion, such as an adhesive overhang region 310 on the backing layer. Water or another biocompatible and appropriate solvent is applied to the permeable region of backing layer 300. The solvent (e.g., water) permeates the backing layer and causes the intermediate layer 200 to dissolve, thereby releasing the microneedles 110 from the device to remain embedded within the skin. The backing layer 300 then may be removed and the residue from the dissolved intermediate layer 200, if any, may be wiped away with a tissue or cloth.

Optionally, the microneedle device may be applied to the skin using an applicator. In this embodiment, the microneedle device is placed on the skin at the desired location. An applicator, such as a roller or flexible pad (e.g., comprising a gel material that can conform to the body surface contours while effectively transferring the applied force to the microneedle device) placed over the user's finger tips to increase the surface area, is then used to apply a greater and/or more even force over the backing layer 300 of the device than may be obtained by pressing the device into the skin using fingertips alone. The use of an applicator may ensure that the microneedles are seated to their full depth before dissolution of the intermediate layer 200 and/or that the device is applied smoothly and tautly to the skin without wrinkles or trapped air bubbles.

Device Manufacture

The inner layer 100 may be manufactured according to any known and appropriate method for manufacturing microneedles. For example, suitable manufacturing methods include wet etching or dry etching using a silicon base, precision machining using metal or resin (electro-discharge machining, laser processing, grinding, hot embossing, injection molding, etc.), and machinery cutting. For embodiments in which hollow microneedles are desired, the microneedles may be hollowed during the molding process or by secondary processing (e.g., by laser cutting).

Other suitable methods for manufacture include centrifuge casting (see, for example, U.S. 2009/0182306) and lithography (see, for example, Moga, et al., Adv. Mater. 2013; DOI:10.102/adma.201300526). In centrifuge casting, a mold is produced by an appropriate technique such as photolithography or by etching in a silicon substrate (e.g., PDMS). An aqueous polymeric solution is then prepared and placed into the mold, preferably as a viscous gel. The filled mold is then centrifuged under conditions that promote filling of the microneedle mold cavities. The filled mold is then dried. Optionally, the mold may be partially filled several times with the same or different polymeric solutions to allow for customization of the microneedles over their length and/or for the incorporation of active ingredients in specific portions/layers of the microneedles. In other casting techniques, the polymer solution is forced into the mold using positive pressure (rollers, e.g. the PRINT process) or negative pressure (vacuum).

The intermediate layer 200 is applied to the inner layer 100 either during or after the manufacturing process, as appropriate. In one example, the intermediate layer 200 is adhered to the fully-formed inner layer 100 by slightly wetting and then drying the water-soluble polymer of the intermediate layer 200 in order to promote adhesion between the layers. Alternatively, a compatible polymer may be used to adhere the two layers. In the case of centrifuge casting, the intermediate layer 200 may be positioned on in or on the mold after the mold is filled but prior to centrifugation.

The outer layer 300 may be affixed to the intermediate layer 200 prior to or after application of the intermediate layer 200 to the inner layer 100.

For embodiments in which active ingredients (e.g., pharmaceutically-active or other cosmetic ingredients) are included in the microneedles 110, those additional ingredients may be added or incorporated by any known and appropriate method. For example, the microneedles 110 may be coated with an active ingredient. Optionally, the coating solution containing a polymer carrier having compatibility with the active ingredient and the microneedles 110, including using the same polymer for both the carrier and the microneedles. Examples of the polymer carrier include pullulan, PEG, carboxyvinyl polymers, PEO, PVP, PVA, cellulose derivatives, and HA and derivatives thereof. Immediately after coating, the coating solution is dried (e.g., by air drying, vacuum drying, freeze drying, or a combination thereof).

Active ingredients that may be delivered to the skin using microneedles include, for example, antioxidants, free radical scavengers, antibacterial agents, antiviral agents, antifungal agents, antihistamines, anti-acne drugs, analgesics, local anesthetics, hair growth-promoting agents, hair growth-inhibiting agents, anti-inflammatory drugs, peptides, polypeptides, proteins, vitamins, amino acids and derivatives, anesthetics, antineoplastic agents, botulinum toxins including botulinum toxin A (e.g., BoTox®, Dysport®, and Xeomin®), B, C, D, E, F, and G, mannitol, oxycodone, bimatoprost, vaccines, lidocaine, sumatriptan, growth factors (e.g., hGH, PDGF, etc.), skin cancer (e.g., melanoma) therapeutics, and mixtures thereof. The active ingredients may be encapsulated into biocompatible and biodegradable microparticles prior to incorporation into the microneedles. The encapsulated or unencapsulated active ingredient may be admixed with the liquid/gel polymer prior to microneedle manufacture.

Exemplary Microneedle Formulations

Formulation 1:

5% Hyaluronic acid (uncrosslinked)

4-5% Pullulan

1-3% Mannitol

Formulation 2:

2.5% Hyaluronic acid (cross-linkable)

5-10% Pullulan

optionally, 1-3% Mannitol

Formulation 3:

5% Hyaluronic acid (uncrosslinked) provided as about 1% high MW HA (e.g., 0.5-2% high MW HA) and qs. with low MW HA

4-5% Pullulan

1-3% Mannitol

The disclosed embodiments are susceptible to various modifications and alternative forms, and specific examples thereof have been shown by way of example in the drawings and are herein described in detail. It should be understood, however, that the disclosed embodiments are not to be limited to the particular forms or methods disclosed, but to the contrary, the disclosed embodiments are to cover all modifications, equivalents, and alternatives.

What is claimed is:

1. A device, comprising:
a first layer comprising a plurality of biocompatible microneedles each having a tip region and a base region;
a second layer comprising a water soluble polymer affixed to the base regions of the microneedles; and
a third layer comprising a water-permeable portion in fluid communication with the second layer.

2. The device of claim 1, wherein the first layer further comprises a substrate layer that is contiguous with the microneedles and attached at the base regions.

3. The device of claim 1, wherein the first layer lacks a substrate layer and the microneedles are attached to the second layer via the respective base regions.

4. The device of claim 1, wherein the microneedles comprise at least one polymer selected from the group consisting of pullulan, hyaluronic acid (HA), polylactic acid (PLA), polyglycolic acid (PGA), poly(lactic-co-glycolic acid) (PLGA), cellulose, sodium carboxymethyl cellulose (SCMC), hydroxyethyl cellulose (HEC), hydroxypropyl cellulose (HPC), hydroxypropyl methylcellulose (HPMC), amylopectin (AMP), hyaluronic acid, silicone, polyvinylpyrolidone (PVP), polyvinyl alcohol (PVA), poly(vinylpyrrolidone-co-methacrylic acid) (PVA-MAA), polyhydroxyethylmethacrylate (pHMEA), polyethlene glycol (PEG), polyethylene oxide (PEO), polyacrylic acid, chrondroitin sulfate, dextrin, dextran, maltodextrin, chitin, chitosan, mono- and polysaccharides, galactose, and maltose.

5. The device of claim 1, wherein the microneedles comprise hyaluronic acid, pullulan, at least one sugar alcohol, or a combination thereof.

6. The device of claim 5, wherein the hyaluronic acid is not crosslinked and is present in a concentration of 3%-6%, and wherein the pullulan is in a concentration of 3%-6%.

7. The device of claim 5, wherein the hyaluronic acid is crosslinked and is present in a concentration of 1%-4%, and wherein the pullulan is in a concentration of 3%-12%.

8. The device of claim 5, wherein the sugar alcohol is mannitol, sorbitol, or xylitol.

9. The device of claim 1, wherein the microneedles further comprise an active ingredient.

10. The device of claim 1, wherein the second layer comprises a polymer selected from the group consisting of pullulan, PVP, PGA, PLGA, PLA, and mixtures thereof.

11. The device of claim 1, wherein the third layer further comprises an overhang region that extends beyond an outer dimension of the first layer and the second layer, and wherein the overhang region further comprises an adhesive on a skin-facing surface.

12. The device of claim 1, wherein the microneedles are disposed on the first layer in a grid-like pattern with regularly spaced rows and columns.

13. A method for delivering a cosmetic polymer to the skin, comprising:
providing a device of claim 1;
applying the device to a skin region of a subject;
applying skin-directed pressure to the third layer sufficient to cause the plurality of microneedles to puncture a stratum corneum of the skin region;
applying a liquid to the third layer to cause the second layer to dissolve;
waiting for a period of time until the second layer is substantially completely dissolved; and
removing the device from the skin region.

14. A device, comprising:
a plurality of biocompatible microneedles,
wherein the microneedles comprise 1.0%-7.5% hyaluronic acid (HA) and 2.5%-15% pullulan.

15. The device of claim 14, wherein the microneedles further comprises a sugar alcohol.

16. The device of claim 15, wherein the sugar alcohol is mannitol.

17. The device of claim 16, wherein the sugar alcohol is present in a concentration of 0.5%-5.0%.

18. A device, comprising:
a plurality of biocompatible microneedles,
wherein the microneedles comprise 1.0%-7.5% hyaluronic acid (HA) and 0.5%-5.0% mannitol.

19. The device of claim 18, wherein the HA is not crosslinked and is present in a concentration of 3%-6%.

20. The device of claim 18, wherein the microneedles comprise crosslinked HA, and wherein the crosslinked HA is present in a concentration of 1%-4%.
